(19) United States
(12) Patent Application Publication
(10) Pub. No.: US 2014/0088353 A1
(43) Pub. Date: Mar. 27, 2014

(54) STEREO ENDOSCOPE APPARATUS AND IMAGE PROCESSING METHOD IN STEREO ENDOSCOPE APPARATUS

(71) Applicant: CANON KABUSHIKI KAISHA, Tokyo (JP)

(72) Inventor: AKIRA HAYAMA, Yokohama-shi (JP)

(73) Assignee: CANON KABUSHIKI KAISHA, Tokyo (JP)

(21) Appl. No.: 14/022,307

(22) Filed: Sep. 10, 2013

(30) Foreign Application Priority Data
Sep. 25, 2012 (JP) 2012-210898

(51) Int. Cl. A61B 1/00; A61B 1/018

(52) U.S. Cl. CPC A61B 1/00009; A61B 1/00193; A61B 1/018
USPC 600/104

(57) ABSTRACT

The stereo endoscope apparatus includes: a treatment instrument that is operable; multiple imaging units for imaging a subject, the multiple imaging units acquiring, in response to an operation of the treatment instrument, an image in which the treatment instrument does not appear and an image in which the treatment instrument appears; a detecting section for detecting the area of the image where the treatment instrument appears; an area determining section for determining a replacement area in which an image is to be replaced, the replacement area being at least a part of the area in which the treatment instrument appears; and an image processing section for replacing the replacement area by the image in which the treatment instrument does not appear.

### BACKGROUND OF THE INVENTION

1. Field of the Invention

The present invention relates to a stereo endoscope apparatus and an image processing method in a stereo endoscope apparatus.

2. Description of the Related Art

A stereo endoscope apparatus includes a pair of left and right optical systems and stereo cameras including image pickup elements at a distal end portion of an endoscope to be inserted in a subject to be observed. The stereo endoscope apparatus picks up left and right images having parallax, which are equivalent to images captured by human eyes, with the pair of left and right optical systems and the stereo cameras, and uses those left and right images to display the subject to be observed in three dimensions on a three-dimensional display apparatus.

FIG. 7 illustrates a configuration of a distal end portion of a conventional stereo endoscope apparatus, in which stereo cameras (101R and 101L), a treatment instrument channel 102 for inserting a treatment instrument, and lighting 103 are arranged. A doctor performs surgery using the treatment instrument and the like while observing the three-dimensional picture shown on the three-dimensional display apparatus.

In such stereo endoscope apparatus, for an object that is brought into close proximity with the stereo cameras, such as the treatment instrument, an angle of convergence determined by the stereo cameras becomes too large, which makes stereoscopy difficult.

To address this problem, Japanese Patent Application Laid-Open No. 2004-65804 discloses a method involving forming a two-dimensional image of only a predetermined area and a method involving generating and superimposing a mask image.

However, the related art disclosed in Japanese Patent Application Laid-Open No. 2004-65804 employs the method involving determining a difficult area for the stereoscopy in advance, and displaying only a monocular image for the area or superimposing the mask image on the area. Therefore, an area in which the two-dimensional image is mixed in the three-dimensional image always exists, which has caused a feeling of interference in the stereoscopy.

### SUMMARY OF THE INVENTION

The present invention enables stereoscopy of most areas and is capable of improving operability of an endoscope by replacing an area (image area) of an object that is brought into proximity with the endoscope not by a two-dimensional image but by a three-dimensional image.

A stereo endoscope apparatus according to one embodiment of the present invention includes: a treatment instrument that is operable; multiple imaging units for imaging a subject, the multiple imaging units acquiring an image in which the treatment instrument does not appear and an image in which the treatment instrument appears in response to an operation of the treatment instrument; a detecting section for detecting an area in which the treatment instrument appears from the image in which the treatment instrument appears; an area determining section for determining a replacement area in which an image is to be replaced, the replacement area being at least a part of the area in which the treatment instrument appears; and an image processing section for replacing the replacement area by the image in which the treatment instrument does not appear.

Further features of the present invention will become apparent from the following description of exemplary embodiments with reference to the attached drawings.

### BRIEF DESCRIPTION OF THE DRAWINGS

FIG. 1 is a functional block diagram of a first embodiment of the present invention.

FIG. 2 illustrates an image processing flow of the first embodiment.

FIGS. 3A, 3B, 3C and 3D are images in processing steps of the first embodiment.

FIG. 4 is a schematic diagram of an endoscope system according to the first embodiment.

FIG. 5 is a schematic diagram of a distal end portion of an endoscope main body according to the first embodiment.

FIGS. 6A, 6B, 6C, 6D, 6E and 6F are images in processing steps of a second embodiment of the present invention.

FIG. 7 is a schematic diagram of a distal end portion of an endoscope main body according to a conventional embodiment.

### DESCRIPTION OF THE EMBODIMENTS

Exemplary embodiments of the present invention are described in detail with reference to the accompanying drawings. It should be noted, however, that the scope of the invention is not limited to the illustrated examples.

### First Embodiment

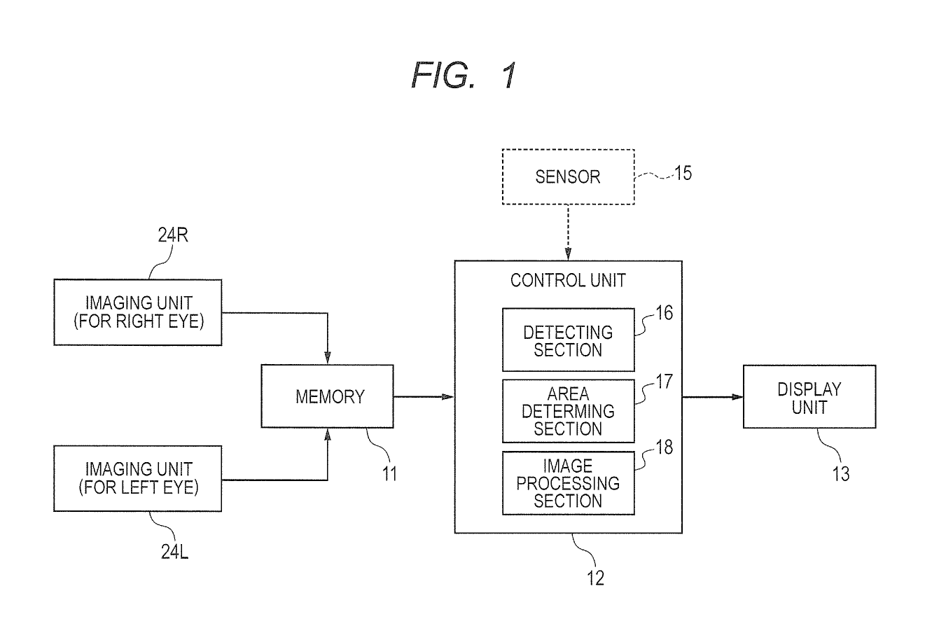

FIG. 1 is a functional block diagram according to a first embodiment of the present invention. FIG. 1 illustrates imaging units 24R and 24L of an endoscope for picking up images of a subject. Further, FIG. 1 illustrates a memory 11, a control unit 12 for detecting a treatment instrument from the picked-up images and replacing a part of an image of the detected treatment instrument by an image in which the treatment instrument does not appear, and a stereo image display unit 13. With this configuration, the respective images captured by the imaging units 24R and 24L are once held in the memory 11. In the control unit 12, a detecting section 16 detects the treatment instrument from the respective images held in the memory 11, an area determining section 17 determines, based on information on the detected treatment instrument, a replacement area in which the image is to be replaced, and an image processing section 18 performs image replacement processing on the replacement area. The obtained result is displayed on the display unit 13 in three dimensions.

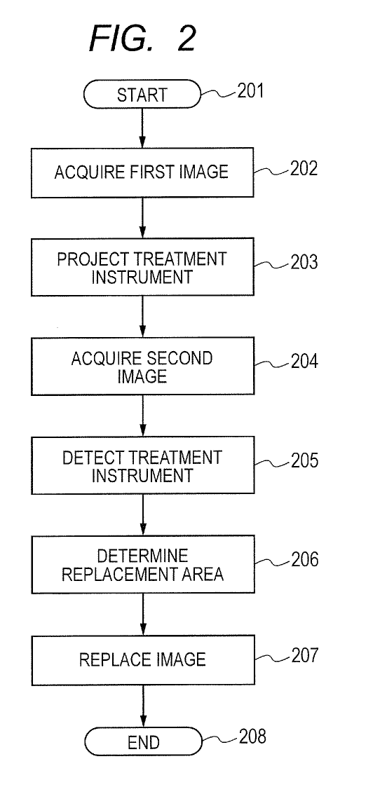

FIG. 2 is a flowchart of image processing of the first embodiment. Hereinafter, the image processing of the first embodiment is described following the flowchart.

In this embodiment, the processing is started in Step 201, and then the processing proceeds to acquisition of a first image of Step 202.

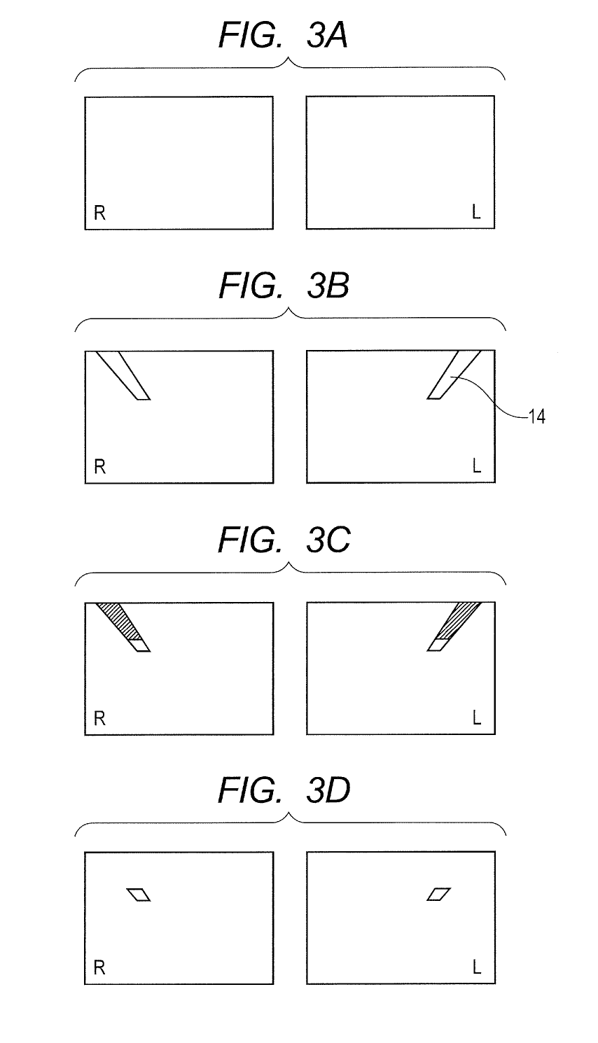

In the acquisition of the first image of Step 202, in a state in which a treatment instrument 14 is not projected from a distal end of the endoscope, imaging is performed with multiple imaging systems. Images obtained as a result are held in the memory 11. FIGS. 3A to 3D are examples of images obtained by the respective imaging systems and held in the memory 11 in processing steps, and FIG. 3A is an example of images obtained in Step 202. In this state, the treatment instrument 14 is not projected from the distal end of the endoscope, and it can be seen that the treatment instrument 14 does not appear in images L and R of FIG. 3A.

After the acquisition of the first image of Step 202 is performed, the processing proceeds to projection of the treatment instrument of Step 203. In the projection of the treatment instrument of Step 203, the treatment instrument 14 is slowly projected from the distal end of the endoscope. At this time, when the treatment instrument 14 is projected abruptly, the treatment instrument is liable to be brought into contact with an organ.

After the projection of the treatment instrument of Step 203, the processing proceeds to acquisition of a second image of Step 204. In the acquisition of the second image of Step 204, in a state in which the treatment instrument 14 is projected from the distal end of the endoscope in response to an operation of the treatment instrument 14, the imaging is performed with the multiple imaging systems. The images obtained as a result are held in the memory 11. FIG. 3B is an example of the images obtained in the acquisition of the second image of Step 204 and held in the memory 11. When stereoscopy is performed in this state, it can be seen that the treatment instrument 14 appears double and causes a feeling of interference.

After the acquisition of the second image is performed in Step 204, the processing proceeds to detection of the treatment instrument of Step 205. In the detection of the treatment instrument of Step 205, an area in which the treatment instrument 14 appears is identified in each of the images acquired in the acquisition of the second image. The identification of the area is performed by analyzing the images obtained in the acquisition of the second image of Step 204 and detecting the area in which the treatment instrument 14 appears. Alternatively, because a trajectory of the treatment instrument 14 is uniquely determined in terms of a structure of the endoscope, a position of the treatment instrument 14 may also be detected by generating a template of the image of the treatment instrument 14 at every position in advance and performing template matching on the trajectory.

After detecting the area in which the treatment instrument 14 appears in the detection of the treatment instrument of Step 205, the processing proceeds to determination of the replacement areas of Step 206. In the determination of the replacement areas of Step 206, it is determined which areas in the area in which the treatment instrument 14 appears are to be replaced. In this embodiment, in order to allow a user to recognize the position of the treatment instrument 14 in performing the treatment, while leaving areas with which a distal end portion of the treatment instrument 14 may be recognized, areas other than the distal end portion of the treatment instrument 14, that is, hatched area in FIG. 3C, are determined as the areas to be replaced. Alternatively, as another embodiment, all areas of the treatment instrument 14 detected in Step 205 are replaced, and the recognition of the treatment instrument 14 may be addressed by placing a marker on a screen. Further, there is a case where double vision due to the proximity occurs in all the areas in which the treatment instrument 14 appears. In such case, for any one of the left and right images, all the areas of the treatment instrument 14 may be determined as the replacement areas, and for the other one of the left and right images, while leaving the distal end portion of the treatment instrument 14, the areas other than the distal end portion of the treatment instrument 14 may be determined as the replacement areas. In this manner, only the distal end portion of the treatment instrument 14 may be recognized as a two-dimensional image, the position of the treatment instrument 14 may be grasped, and the double vision due to the proximity may be prevented.

After the determination of the replacement areas of Step 206 is performed, the processing proceeds to replacement of the images of Step 207. In the replacement of the images of Step 207, the replacement areas determined in Step 206 are replaced by images of corresponding areas of the images obtained in the acquisition of the first image of Step 202, where the corresponding areas correspond to the replacement area. In this manner, the double vision due to the proximity of the treatment instrument 14 does not occur, and the stereoscopy may be performed in most areas of the screen, which enables observation with the endoscope with higher operability. FIG. 3D illustrates images after the replacement. At this time, the images acquired by the acquisition of the first image of Step 202 are not limited to the images immediately preceding the projection of the treatment instrument of Step 203. For example, images acquired prior thereto may also be used.

After the replacement of the images of Step 207, the processing proceeds to Step 208, in which the image processing ends.

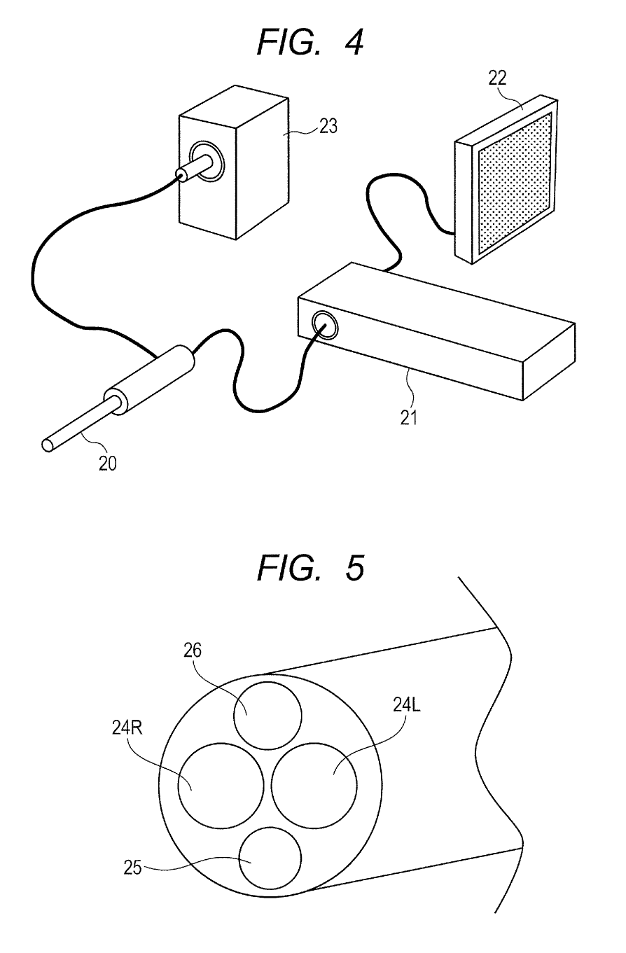

Next, an embodiment of an endoscope system to which the present invention is applied is described with reference to FIG. 4. In this example, the endoscope system includes an endoscope main body 20, a control box 21 detachably attachable to the endoscope main body, a monitor display 22 connected to the control box 21, and a light source 23. Further, a distal end of an insertion portion of the endoscope main body according to this embodiment is illustrated in FIG. 5. Each of the imaging units 24R and 24L includes a lens system and an image sensor such as a charge coupled device (CCD) or a complementary metal oxide semiconductor (CMOS) (not shown). Further, in the distal end of the insertion portion, an illumination outlet 25, and a channel aperture 26 for the treatment instrument that is operable are arranged. In the control box 21, the memory and the control unit of the above-mentioned functional block diagram (FIG. 1) are arranged.

As described above, in this embodiment, areas in which the treatment instrument appears in the images acquired by the respective imaging units are detected, and a part or all of the areas are replaced by images at the same positions in the state in which the treatment instrument does not appear. In this manner, in the observation with the stereo endoscope, even when there is an object that is brought into proximity with the endoscope, the observation with the three-dimensional image without the double vision due to the proximity is enabled in the most areas of the screen, and the stereo endoscope with higher operability may be provided.

### Second Embodiment

A second embodiment of the present invention addresses a case where the endoscope is moved when the treatment instrument 14 is projected. In this case, a problem occurs in which, when the corresponding areas of the images acquired in the acquisition of the first image of Step 202 are directly used in the replacement of the images of Step 207 as in the first embodiment, misalignment occurs. To address this problem, in this embodiment, in the replacement of the images of Step 207, images of the corresponding areas, which correspond to the replacement areas determined in the determination of the replacement areas of Step 206, are determined by matching between the images acquired in the acquisition of the first image of Step 202 and the images acquired in the acquisition of the second image of Step 204. Thereafter, the replacement areas are replaced by using the images of the corresponding areas in the images acquired in the acquisition of the first image of Step 202, which are determined by the matching.

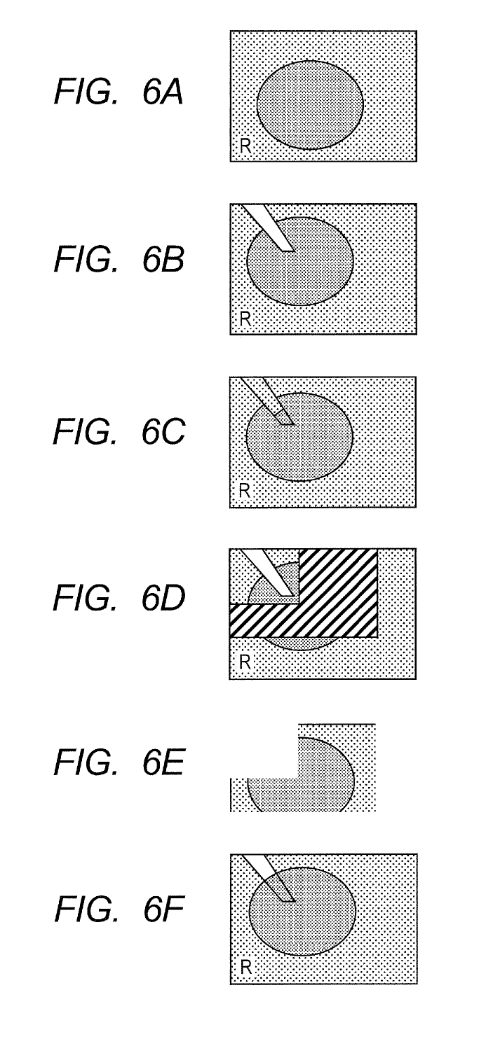

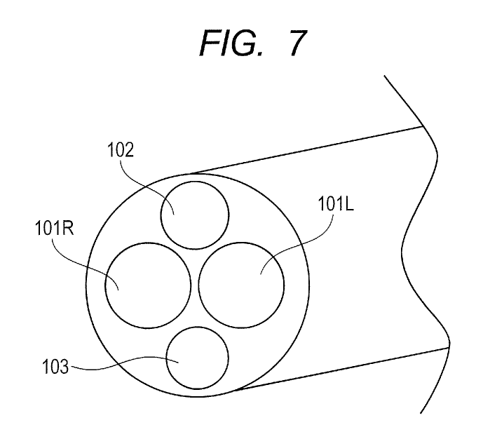

Next, a specific method of the matching is described in detail. FIG. 6A is an image acquired by the right imaging system in the acquisition of the first image of Step 202. In contrast, FIG. 6B is an image acquired in the acquisition of the second image of Step 204 in the state in which the treatment instrument 14 is projected, but it can be seen that due to the displacement of the endoscope, as compared to the subject in the image of FIG. 6A, the subject in the image is slightly shifted. Hereinafter, processing on the image acquired by the left imaging system is similarly performed, and is therefore omitted. An image obtained in a case where the processing according to the first embodiment is applied to the image is illustrated in FIG. 6C, and in such the image, the image of the replacement area after the replacement is shifted. This is because the image acquired in the acquisition of the first image of Step 202, which corresponds to the area to be replaced, is shifted. Therefore, in this embodiment, the corresponding area in an image before the treatment instrument is projected, which corresponds to the area to be replaced, is detected. To be specific, an area in which the treatment instrument 14 does not appear in a range that is slightly narrower than the original image in the image acquired in the acquisition of the second image (FIG. 6B) is used as a template, and region-based matching between the template and the image acquired in the acquisition of the first image (FIG. 6A) is performed. The specific range is the hatched area of FIG. 6D, and the image is as illustrated in FIG. 6E. As a degree of difference of the region-based matching, a sum of absolute differences (SAD) may be used. The SAD is a sum of absolute values of differences of luminance values of pixels of the template image and the image to be searched at the same position, which is an index indicating that, as the value becomes closer to 0, the template image and the image to be searched are more similar, and is defined by the following equation:

$${R}_{SAD} = {\sum }_{( i , j ) = ( 1 , 1 )}^{( {N}_{1} , {N}_{2} )} \vert I ( i , j ) - T ( i , j ) \vert$$

In the equation, (N₁,N₂) is a size of the template, T(i,j) is the template, and I(i,j) is the image to be searched. In reality, the SAD is computed while moving the template with respect to the image (FIG. 6A) acquired in the acquisition of the first image to find a position at which the SAD becomes the smallest. In this manner, the corresponding area in the image acquired in the acquisition of the first image of Step 202, which corresponds to the replacement area determined in the determination of the replacement area of Step 206, may be determined. Thereafter, the corresponding area is used to perform the replacement of the image of Step 207 so that the replacement may be performed as in FIG. 6F without the misalignment.

The template is not necessarily limited to that used in this embodiment, and the template may be the entire image, or a rectangular area, a circular area, or other polygonal area as a part cut out from the image. Further, for the region-based matching, methods such as a sum of squared differences (SSD), normalized cross correlation (NCC), and phase-only correlation (POC) may be used. Further, as in the first embodiment, the images acquired by the acquisition of the first image of Step 202 are not limited to the image immediately preceding the projection of the treatment instrument of Step 203. For example, when an image having a positional relationship that is closer to the image acquired in Step 204 than the immediately preceding image is available, the image may be used.

In this embodiment, even when the endoscope is displaced between the state in which the endoscope is projected and the state in which the endoscope is not projected, the region-based matching processing is performed to determine an amount of displacement so that the image for replacing the area of the treatment instrument 14 may be determined appropriately to perform the replacement of the image. In this manner, in the observation with the stereo endoscope, even when there is an object that is brought into proximity with the endoscope, the observation with the three-dimensional image without the double vision due to the proximity is enabled in most of the screen, and the stereo endoscope with higher operability may be provided.

### Third Embodiment

In a third embodiment of the present invention, in the detection of the treatment instrument of Step 205, instead of extracting the treatment instrument 14 from the image acquired in the acquisition of the second image of Step 204, a feed of the treatment instrument is detected by a sensor 15 to detect how much the treatment instrument 14 is projected from the distal end of the endoscope.

Now, the third embodiment is described in detail with reference to FIG. 1. In the third embodiment, the sensor 15 is further provided. The sensor 15 detects the feed of the treatment instrument 14, and notifies the control unit 12 of information on the detected feed of the treatment instrument 14. In this manner, the detecting section 16 in the control unit 12 detects, based on the information from the sensor, the area in which the treatment instrument appears in the image, the area determining section 17 determines, based on the information from the detecting section 16, the replacement area in which the image is to be replaced, and the image processing section 18 performs the image replacement processing on the replacement area. The obtained result is displayed on the display unit 13 in three dimensions.

In the first embodiment, as described above, in the detection of the treatment instrument of Step 205, the detecting section 16 analyzes the image acquired in the acquisition of the second image of Step 204 to extract the area in which the treatment instrument 14 appears. In contrast, in the third embodiment, based on the information on the feed of the treatment instrument from the sensor, the area in which the treatment instrument appears in the image is detected.

In this embodiment, the feed of the treatment instrument is detected by the sensor so that the projection of the treatment instrument 14 may be detected accurately, and hence the area in which the image is to be replaced may be determined appropriately to perform the replacement of the image. In this manner, in the observation with the stereo endoscope, even when there is an object that is brought into proximity with the endoscope, the observation with the three-dimensional image without the double vision due to the proximity is enabled in most of the screen, and the stereo endoscope with higher operability may be provided.

According to the present invention, in the observation with the stereo endoscope, even when there is an object that is brought into proximity with the endoscope, the observation with the three-dimensional image without the double vision due to the proximity is enabled in the most areas of the screen, and the stereo endoscope with higher operability may be provided.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-210898, filed Sep. 25, 2012, which is hereby incorporated by reference herein in its entirety.

What is claimed is:

1. A stereo endoscope apparatus, comprising:
a treatment instrument that is operable;
multiple imaging units arranged to image a subject, the multiple imaging units acquiring an image in which the treatment instrument does not appear and an image in which the treatment instrument appears in response to an operation of the treatment instrument;
a detecting section configured to detect an area in which the treatment instrument appears from the image in which the treatment instrument appears;
an area determining section configured to determine a replacement area in which an image is to be replaced, the replacement area being at least a part of the area in which the treatment instrument appears; and
an image processing section configured to replace the replacement area by the image in which the treatment instrument does not appear.

2. A stereo endoscope apparatus according to claim 1, wherein the image processing section detects a corresponding area in the image in which the treatment instrument does not appear that corresponds to the replacement area, and replaces the replacement area by an image of the corresponding area.

3. A stereo endoscope apparatus according to claim 2, wherein the image processing section detects the corresponding area by region-based matching between the image in which the treatment instrument does not appear and the image in which the treatment instrument appears.

4. A stereo endoscope apparatus according to claim 1, further comprising a sensor for detecting a feed of the treatment instrument,
wherein the detecting section detects the area in which the treatment instrument appears based on the feed of the treatment instrument detected by the sensor.

5. A stereo endoscope apparatus according to claim 1, wherein the detecting section performs template matching on a trajectory of the treatment instrument by using a template of an image of the treatment instrument, which is generated in advance, to detect the area in which the treatment instrument appears.

6. A stereo endoscope apparatus according to claim 1, wherein the area determining section determines, in order to allow a user to recognize the treatment instrument, an area other than a distal end portion of the treatment instrument in the area in which the treatment instrument appears as the replacement area.

7. A stereo endoscope apparatus according to claim 1,
wherein the replacement area is an entirety of the area in which the treatment instrument appears, and
wherein the image processing section places a marker of the treatment instrument in an image after the replacement in order to allow a user to recognize the treatment instrument.

8. A stereo endoscope apparatus according to claim 1, wherein the area determining section determines, in order to allow a user to recognize the treatment instrument, for an image acquired by one of the multiple imaging units, an area other than a distal end portion of the treatment instrument in the area in which the treatment instrument appears as the replacement area, and for images acquired by others of the multiple imaging units, an entirety of the area in which the treatment instrument appears as the replacement area.

9. An image processing method in a stereo endoscope apparatus, comprising:
acquiring, by multiple imaging units, images in which a treatment instrument does not appear;
operating the treatment instrument;
acquiring, by the multiple imaging units, images in which the treatment instrument appears in response to the operation of the treatment instrument;
detecting an area in which the treatment instrument appears from the image in which the treatment instrument appears;
determining a replacement area in which an image is to be replaced from the area in which the treatment instrument appears; and
replacing the replacement area by the image in which the treatment instrument does not appear.

10. An image processing method according to claim 9, further comprising detecting a corresponding area in the image in which the treatment instrument does not appear that corresponds to the replacement area,
wherein the replacing comprises replacing the replacement area by an image of the corresponding area.

11. An image processing method according to claim 10, wherein the detecting of the corresponding area comprises detecting the corresponding area by region-based matching between the image in which the treatment instrument does not appear and the image in which the treatment instrument appears.

12. An image processing method according to claim 9, wherein the detecting of the area in which the treatment instrument appears comprises detecting the area in which the treatment instrument appears from the image in which the treatment instrument appears based on a feed of the treatment instrument detected by a sensor.

13. An image processing method according to claim 9, wherein the detecting of the area in which the treatment instrument appears comprises performing template matching on a trajectory of the treatment instrument by using a template of an image of the treatment instrument, which is generated in advance, to detect the area in which the treatment instrument appears.

14. An image processing method according to claim 9, wherein the determining of the replacement area comprises determining, in order to allow a user to recognize the treatment instrument, an area other than a distal end portion of the treatment instrument in the area in which the treatment instrument appears as the replacement area.

15. An image processing method according to claim 9,
wherein the replacement area is an entirety of the area in which the treatment instrument appears, and
wherein the replacing comprises placing a marker of the treatment instrument in an image after the replacing in order to allow a user to recognize the treatment instrument.

16. An image processing method according to claim 9, wherein the determining of the replacement area comprises determining, in order to allow a user to recognize the treatment instrument, for an image acquired by one of the multiple imaging units, an area other than a distal end portion of the treatment instrument in the area in which the treatment instrument appears as the replacement area, and for images acquired by others of the multiple imaging units, an entirety of the area in which the treatment instrument appears as the replacement area.
